(19) United States
(12) Patent Application Publication
(10) Pub. No.: US 2018/0333472 A1
(43) Pub. Date: Nov. 22, 2018

(54) TREATMENT OF PLANTAR FASCIITIS

(71) Applicant: DT SCIMED, LLC, Towson, MD (US)

(72) Inventor: L. Andrew Koman, Winston-Salem, NC (US)

(73) Assignee: DT SCIMED, LLC, Towson, MD (US)

(21) Appl. No.: 15/978,931

(22) Filed: May 14, 2018

Related U.S. Application Data
(60) Provisional application No. 62/507,628, filed on May 17, 2017.

(51) Int. Cl. A61K 38/48; A61K 9/00

(52) U.S. Cl. CPC A61K 38/4893; A61K 9/0019

(57) ABSTRACT

A novel dosing treatment for the administration of botulinum toxin injected into the calf muscle, namely, gastrocnemius-soleus complex in a range of about 50 to about 180 units and foot muscle group comprising one or more of a group consisting of the plantar intrinsic muscles, the plantar fascia muscles and calcaneal periosteum muscles in a range of about 20 to about 90 units of a patient to treat plantar fasciitis.

### RELATED APPLICATIONS

This is a utility patent application claiming priority and benefit from U.S. Provisional Patent Application No. 62/507,628, filed May 17, 2017.

### STATEMENT REGARDING FEDERALLY SPONSORED RESEARCH OR DEVELOPMENT

None

### REFERENCE TO SEQUENCE LISTING, A TABLE OR A COMPUTER PROGRAM LISTING COMPACT DISC APPENDIX

None.

### BACKGROUND OF THE INVENTION

### 1. Field of Invention

This invention relates to a dosing protocol for the administration of botulinum toxin as a treatment of plantar fasciitis and other conditions.

### 2. Background of the Invention

Plantar fasciitis is a painful enthesopathy or inflammatory process of the plantar fascia (the connective tissue on the sole (bottom surface) of the foot and its origin from the calcaneal periosteum. It may be often caused by overuse of the plantar fascia or arch tendon of the foot. Plantar fasciitis is a common condition and some cases are difficult to treat and require surgery.

Typical treatments for plantar fasciitis may include rest, weight avoidance, weight loss, stretching, orthotics, oral anti-inflammatories, injections of corticosteroids; and invasive surgery. These types of treatments may involve the application of drugs, therapy, or surgery.

Approximately 10% of the general population experiences heel pain or plantar fasciitis during their lifetime. Heel pain is the most common complaint of patients to podiatrists and orthopaedic foot and ankle surgeons. Plantar fasciitis, the most common etiology of heel pain, causes significant discomfort and negatively impacts the health-related quality of life of affected individuals.

Plantar fasciitis is a self-limiting condition for many patients. Approximately, 10% to 20% of patients continue to experience symptoms for six (6) to nine (9) months or more. The cause of plantar fasciitis is multifactorial and incompletely elucidated (League, 2008; Monteagudo, 2013). Age, high body mass index, abnormal foot posture, use of poor foot wear, and repetitive trauma have all been suggested as risk factors. Treatment options include: leg/foot stretching exercises, manual therapy, taping, night splints, electrotherapy, phonophoresis, ultrasound, shoe inserts, heel pads, steroid injections, botulinum toxin injections, extracorporeal shock wave therapy, platelet rich plasma injections, and surgery (Martin, 2014).

Botulinum toxin was theorized over a decade ago to have potential efficacy in the management of plantar fasciitis (Seyler, 2008). The results of five clinical trials (Babcock, 2005; Diaz-Llopis, 2012; Diaz-Llopis, 2013; Elizondo-Rodriguez, 2013; Huang, 2010) have documented the potential efficacy of botulinum toxin in managing the symptoms of plantar fasciitis.

Botulinum toxins, in particular Botulinum Toxin type A, have been used in the treatment of a number of neuromuscular disorders and conditions involving muscular spasm as well as in cosmetic procedures; for example, strabismus, blepharospasm, spasmodic torticollis (cervical dystonia), oromandibular dystonia and spasmodic dysphonia (laryngeal dystonia). The toxin cleave (rendering them inactive) SNARE proteins which are responsible for the attachment of presynaptic vesicles containing neurotransmitters to the presynaptic membrane. In the case of muscles, active toxin rapidly and strongly attaches to neuromuscular junctions preventing presynaptic vesicles from releasing acetylcholine molecules thereby reducing or eliminating the activation of postsynaptic muscles, nerves, or effector tissues. This results in local paralysis and hence relaxation of the muscle afflicted by spasm. Similarly with painful and inflamed connective tissue, pain mediators such as substance P and glutamate are decreased and nutritional blood flow is improved by decreasing alpha adrenergic transmitters within microvascular beds.

The term botulinum toxin as used herein is a generic term embracing the family of toxins produced by the anaerobic bacterium Clostridium botulinum and, to date, seven immunologically distinct toxins have been identified. These have been given the designations A, B, C, D, E, F and G. Further information concerning the properties of the various botulinum toxins, discussed in the article by Jankovic & Brin, The New England Journal of Medicine, No. 17, 1991, pp. 1186-1194 and to the review by Charles L Hatheway, Chapter 1 of the book entitled Botulinum Neurotoxin and Tetanus Toxin, 1^{st} Ed., Lance Simpson, published by Academic Press Inc. of San Diego, Calif., 1989, the disclosures in which are incorporated herein by reference.

The neurotoxic component of botulinum toxin has a molecular weight of about 150 kilodaltons (kD) and is comprised of a short polypeptide chain of about 50 kD which is considered to be responsible for the toxic properties of the toxin, and a larger polypeptide chain of about 100 kD which is believed to be necessary to enable the toxin to penetrate the nerve. The “short” and “long” chains are linked together by means of disulphide bridges.

Therapeutic use of these toxins represents a somewhat unique pharmacokinetic profile. In order for toxin to produce its desired action, it must not only be delivered to the target tissue, e.g. muscle (usually by direct injection), but it must also bind to terminal portions of nerves innervating the target tissue (i.e. the neuromuscular junction), and be transported across the presynaptic terminal membrane into the intracellular domain where the active molecule is cleaved from the binding portion of the divalent complex. Then the active molecule must bind and enzymatically inactivate molecules in the nerve terminal specific for neurochemical transmission. Thus, the toxin molecules are not delivered systemically to distribute throughout the body. The ultimate target is not a specific muscle or organ but rather molecules located in specific nerves which innervate the target tissue within an anatomically defined region of the target tissue or muscle. For example, within skeletal muscle fibers, nerves are not uniformly distributed through the muscle but are restricted to the neuromuscular juncture region of the muscle. In the case of muscle fibers, prior research has shown that different muscles have different numbers of neuromuscular junctions and the total number of these neuromuscular junctions is not dependent on the mass or volume of the muscle or the individual but rather on other factors such as the function of the muscle fibers.

The goal for each of these uses of botulinum toxins is that the toxin be administered in a dosage and volume appropriate to achieving the desired response while remaining localized within the desired specific region of injection. Because the ultimate site of toxin action is nerve junctions within certain regions of the target tissue, over- and under-dosing remains a significant challenge. Administration of too high an absolute dose (total number of toxin molecules relative to the total number of neuromuscular junction targets) or too high a volume of injection might produce adverse reactions related to diffusion of the toxin. Diffusion of the toxin into undesired areas can produce inappropriate paralysis or pathophysiological responses. Too high a dose will produce the desired effect of tissue paralysis but also result in toxin distribution to non-targeted tissues thereby causing an unintended loss of physiological function in these regions. Additionally, delivery of supraoptimal toxin doses presents an undesired immunological challenge which may cause reduced effectiveness on subsequent administrations of the toxin.

Pain from plantar fasciitis, an inflammatory enthesopathy, is produced by nociceptive areas within the plantar fascia and the periosteal attachment to the calcaneus. Pain is exacerbated by contracted plantar fascia, contracted muscles in the foot and shortened gastrocnemius and soleus calf muscles.

Botulinum toxins decrease nociceptors within the periosteum and plantar fascia by preventing neurotransmitters/receptors medicated pain mechanism (e.g., blocking substance p or glutamate) and dysfunctioning (relaxing) short muscles in the foot and calf by deactivating neuromuscular junctions within the gastrocnemius and soleus.

### SUMMARY OF THE INVENTION

The present invention is a novel dosing method using botulinum toxin for the treatment of plantar fasciitis. The treatment includes determining the target muscles in the foot, namely, the plantar intrinsic muscles, the plantar fascia and calcaneal periosteum that produce pain and increase forces in the foot, determining the distribution and location of neuromuscular junctions in the target muscle, and injecting an appropriate therapeutic dose of botulinum toxin in the target muscle in the bottom of the foot. A second therapeutic dose of botulinum toxin is injected in the gastrocnemius-soleus complex (tricep surae) muscles in the calf. The dosing regimen in the aforementioned tissues ensures efficacy, while minimizing possible side effects and cost by ensuring that only that amount of toxin necessary to achieve the desired effect is used.

It is an object of this invention to provide a safe dosing method using botulinum toxins for the treatment of plantar fasciitis; and

It is another object of this invention to provide a simple, easily complied with dosing method of Botulinum Toxins A and B through F in the treatment of plantar fasciitis.

It is yet another object of the invention to address the pain focus while decreasing forces that prevent healing and increase pain.

### BRIEF DESCRIPTION OF THE DRAWINGS

FIG. 1 illustrates parts of the plantar fascia in a dissected bottom view of the human foot showing representative injection sites for the foot muscles; and

FIG. 2 is a diagram of a human leg and foot and the calf muscle of the leg showing representative injection sites for the calf muscles;

These and other objects, advantages, and novel features of the present invention will become apparent when considered with the teachings contained in the detailed disclosure along with the accompanying drawings.

### DESCRIPTION OF THE INVENTION

While the invention is described in connection with certain preferred embodiments, it is not intended that the present invention be so limited. On the contrary, it is intended to cover all alternatives, modifications, and equivalent arrangements as may be included within the spirit and scope of the invention as defined by the appended claims.

The invention is a novel dosing method for injecting Botulinum Toxin, preferably Botulinum Toxin A and/or Botulinum Toxin B or Botulinum Toxin C, D, E and F in the foot and calf muscle of the patient to treat the condition known as plantar fasciitis.

As noted below, at least two types of botulinum toxin, types A and B, are available commercially in formulations for treatment of certain conditions. The term “botulinum toxin” as used herein is meant to refer to any of the known types of botulinum toxin, whether produced by the bacterium or by recombinant techniques, as well as any such types that may be subsequently discovered including engineered variants or fusion proteins. As mentioned above, at the present time, six immunologically distinct botulinum neurotoxins have been characterized, namely botulinum neurotoxin serotypes A, B, C, D, E and F, each of which is distinguished by neutralization with type-specific antibodies. The botulinum toxin serotypes C-F are available from Sigma-Aldrich and from Metabiologics, Inc. (Madison, Wis.), as well as from other sources. The different serotypes of botulinum toxin vary in the animal species that they affect and in the severity and duration of the paralysis they evoke.

Although all botulinum toxin serotypes interfere with proteins that cause the exocytosis of synaptic vesicles, the each interfere with different proteins or different parts of the same protein. These neurotoxins are serologically related but distinct although the molecular weight 150 kD is about the same.

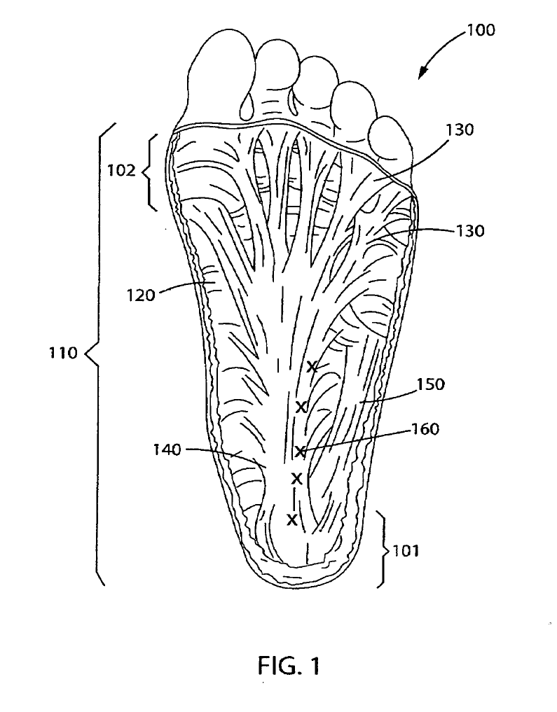

FIG. 1 is a dissected bottom view of a human foot 100 provided to illustrate some of the parts of a plantar fascia 110 located in the bottom of the human foot 100. As shown in FIG. 1, the plantar fascia 110 extends from about the location of the heel 101 to about the location of the ball 102 of the foot. The plantar fascia 110 includes medial plantar fascia 120, superficial tracts 130, a central component of the plantar fascia 140, and a lateral component of the plantar fascia 150. The separate portions of the plantar fascia 110 act as a shock absorber while walking and transfer tensile forces along the bottom of the foot 100.

The plantar fascia 110 serves the vital role of maintaining the shape of the two anatomical arches of the foot, the transverse arch and the longitudinal arch. As longitudinal and lateral tensile stresses are produced in the bottom of the foot 100, the plantar fascia 110 absorbs the tensile forces and maintains the shape of the two anatomical arches.

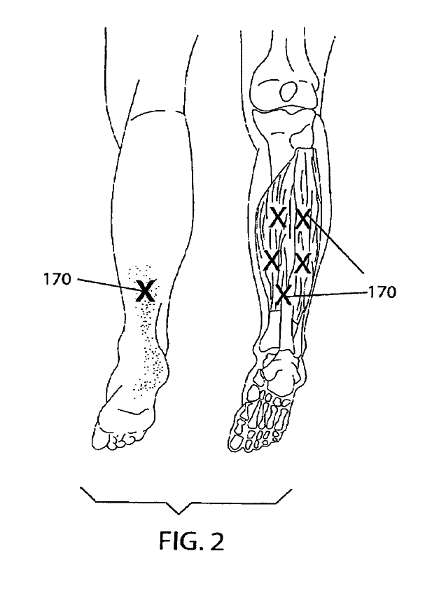

If the tension on the plantar fascia 110 becomes excessive, the plantar fascia 110 may be damaged and produce a condition called plantar fasciitis. Plantar fasciitis is a painful medical condition resulting from inflammation of the plantar fascia 110. The plantar fascia 110 is thick and essentially inelastic. Overstressing the plantar fascia 110 may produce tears in the plantar fascia or separate the plantar fascia 110 from bone and other surrounding materials. Tearing and separation of the plantar fascia 110 produces the painful inflammation known as plantar fasciitis. Frequently, the inflamed areas are along the arch of the foot 100 or near the heel 101 of the foot 100 as shown in FIG. 2.

Plantar fasciitis may be quite debilitating in that everyday activities such as walking and standing are painful.

Botulinum toxin A is produced by Allergan Pharmaceuticals as BOTOX®; by Ipsen Pharmaceuticals, Ltd. as DYSPORT®; and by Merz as Xeomin®. Each vial of BOTOX® and Xeomin® (incobotulinumtoxinA) contains 100 units of C. botulinum type A neurotoxin complex, 0.5 mg of human albumin, and 0.9 mg of sodium chloride as a vacuum-dried frozen powder that requires reconstitution. One unit of both is equal to the median intraperitoneal lethal dose (LD50) in Swiss-Webster mice weighing 18 to 20 g. The LD50 for Botox® has been calculated in primates at 39 to 56 units/kg body wt. However, the exact lethal dose in humans is unknown. The calculated human LD50 of 59 units is based on an extrapolation of data.

DYSPORT® clostridium botulinum type A toxin-hemogglutinin complex is available in 500-unit vials. DYSPORT® units of activity equal 1 mouse LD50 based on their specific assay technique and is sometimes referred to in nanograms, with 1 nanogram equal to 40 units. In the United Kingdom and many other countries, it is approved and labeled for multiple indications, including spasticity of the arm in patients following stroke, dynamic equinus foot deformity due to spasticity in ambulant pediatric cerebral palsy patients, two years of age or older, spasmodic torticollis, blephorospasm, and hemifacial spasm.

Botulinum toxin B is produced by Soltice Neurosciences as MYOBLOC® (rimabotulinumtoxinB). Each injection is a sterile liquid formulation of a purified neurotoxin that acts at the neuromuscular junction to produce flaccid paralysis. MYOBLOC® is provided as a clear and colorless to light-yellow sterile injectable solution in 3.5-mL glass vials. Each single-use vial of formulated MYOBLOC® contains 5,000 Units of Botulinum Toxin type B per milliliter in 0.05% human serum albumin, 0.01 M sodium succinate, and 0.1 M sodium chloride at approximately pH 5.6. One unit of MYOBLOC® corresponds to the calculated median lethal intraperitoneal dose (LD50) in mice. The method for performing the assay is specific to Solstice Neurosciences' manufacture of MYOBLOC. Due to differences in specific details such as the vehicle, dilution scheme and laboratory protocols for various mouse LD50 assays, units of biological activity of MYOBLOC® cannot be compared to or converted into units of any other botulinum toxin or any toxin assessed with any other specific assay method. Therefore, differences in species sensitivities to different botulinum neurotoxin serotypes preclude extrapolation of animal dose-activity relationships to human dose estimates. The specific activity of MYOBLOC® ranges between 70 to 130 Units/ng. MYOBLOC® to Botox is 30-50::1

BOTOX®, DYSPORT®, XEOMIN® are reconstituted in injectable physiologic saline prior to intramuscular injection. MYOBLOC® comes premixed in 3.5-mL glass vials. Each single-use vial of formulated MYOBLOC contains 5,000 Units of botulinum toxin type B per milliliter in 0.05% human serum albumin, 0.01 M sodium succinate, and 0.1 M sodium chloride at approximately pH 5.6.

One unit but have the volume adjusted. Both the volume of fluid and number of units of drug must be considered when preparing the toxin for injection. Dosage is defined in absolute terms, based on the number of units per target muscle diluted to volume based on the size of the structure to be injected and quantity and distribution of neuromuscular junctions. The number of units to be injected is calculated by the quantity of SNARE containing organelles (e.g., neuromuscular junctions) to be neutralized, and the volume is determined by the mass of the target muscle, and the number of injection sites by the anatomic distribution of the neuromuscular junctions. Once the appropriate number of active toxin molecules (units) for a given muscle is determined, the dose in units remains constant and the volume and number of injection sites is adjusted based upon growth and anatomy. For example, there are an estimated pikamole of active toxin molecules in 100 units of BOTOX®. Hence, there are sufficient active toxin molecules to block effectively all neuromuscular junctions of the “target” muscle. The toxin is thereafter injected within the muscle or skin as close to the neuromuscular junctions (or other SNARE-containing organelle) of the calf and foot as possible using palpation and anatomic landmarks, fluoroscopy and/or ultrasonography to localize their position. Electromyography cannot localize directly the plantar fascia or periosteum.

Visualization of calf and foot muscles can be performed reliably using linear probe ultrasonography (ultrasound) with a frequency ranging from 5-18 Mhz. For injection localization, linear beam applications better define and delineate the anatomic relationships between muscles, tendons or bones. Higher frequencies are recommended for the localization of the superficial muscles or layers, while lower frequencies may be used for deep structures. The muscles are covered by the epimysium which is the connective tissue that surrounds the entire muscle. The epimysium extends into the muscle to become the perimysium, which divides the fascicle into muscle fibers. The perimysium and the muscular fascicles can be identified because the muscular bundles are hypoechoic (less bright) while the epimysium and perimysium appear as hyperechoic structures. On longitudinal scanning, the fascia is depicted as a fibrillar hyperechoic sheath surrounding the muscle.

Injection Technique

Botulinum toxin dose for treatment plantar fasciitis are applicable to Botulinum toxin A, B and other toxins such as Botulinum toxins C-F. Equivalencies are BOTOX® (1 unit) equals RT002 (1 unit) equals Xeomin®, DYSPORT® (2 to 5 units) equals MYOBLOC® (25 to 30 units).

In a Preferred Treatment:

Two (2) vials (vial A and B) of Daxibotulinumtoxin A or Botulinum Toxin B for Injection or placebo (160 units/vial) powder are each reconstituted with 5 (five) mL of sterile saline. The injection product is prepared for administration by withdrawing from vial A five (5) mL (150-160 U) of the solution into a syringe fitted with a 27 g×1.5 in needle. Vial A containing five (5) mL (160 U) of the reconstituted solution are injected into the gastrocnemius-soleus complex (triceps surea) in five (5) to seven (7) injection sites 160 (FIG. 2) depending on leg length/mass and neuromuscular junctions (NMJ) distribution.

Supplemented by the almost vestigial plantaris muscle, the two heads of the gastrocnemius muscle and the soleus muscle form the triceps surea. The gastrocnemius muscle forms the greater bulk of the calf. It arises by two heads. The larger medial head takes origin from the rough area of the popliteal surface of the femur immediately above the medial femoral condyle. The lateral head arises from an impression on the upper and posterior part of the lateral surface of the lateral femoral condyle and from the distal end of the lateral supracondylar line of the femur. The soleus is a broad, fleshy muscle lying immediately anterior to the gastrocnemius but arising entirely below the knee.

From vial B, two and half (2.5) mL (80 U) are withdrawn into a syringe fitted with a 27 g×1.5 in needle for injection into multiple sites 170 (FIG. 1) the plantar intrinsic muscles, plantar fascia and calcaneal periosteum.

The dose in units and the volume were derived to optimize active toxin molecule delivery to “target” receptor/neurotransmitter containing muscle or fascia/periosteal nociceptors based upon dose dilution distribution principles published by Koman et al. (U.S. Pat. Nos. 8,632,768 and 8,506.970) which are incorporated herein by reference. The dose remains constant regardless of weight, height or muscle size but the volume is adjusted in the calf.

In the following dosage treatment, the term “selected calf muscle” refers to the gastrocnemius-soleus complex (tricep surae) muscles and the term “selected foot muscle” refers to the plantar intrinsic muscles, plantar fascia and calcaneal periosteum.

Using 1 unit base equals BOTOX®; Xeomin®; Revance RT002 alternative dose treatment ranges of the present invention are:

a. based upon dose dilution target organ, another treatment dosage would be 100 to 108 units base in 5 cc saline solution in the selected calf muscle and 30 to 40 units base in 2.5 cc saline solution in the selected foot muscle. DYSPORT® 2.5 (same volume) and MYOBLOC® 25 (same volume)

b. selected calf muscle 50 unit base diluted in 8 cc saline solution to the selected foot muscle 20 unit base diluted to in 3 cc saline solution;

c. selected calf muscle 100 unit base diluted in 6 cc saline solution to the selected foot muscle 30 unit base diluted in 2.5 cc saline solution;

d. selected calf muscle 150 unit base diluted in 5 cc saline solution to the selected foot muscle 40 unit base diluted in 2.5 cc saline solution;

e. selected calf muscle 200 unit base diluted in 5 cc saline solution to the selected foot muscle 50 unit base diluted in 2 cc saline solution; and

f. selected calf muscle 300 unit base diluted in 2.5 cc saline solution to the selected foot muscle 60 unit base diluted in lcc saline solution.

It was found that botulinum toxin doses up to 200 units could be injected into a selected calf muscle and doses up to 90 units could be injected into a selected foot muscle.

Alternatively, a treatment method may be used where the botulinum toxin serotype is injected solely in the foot muscles, quadratus plantae and short flexor. The dosage used is more fully described in U.S. Pat. No. 8,506,970 issued Aug. 13, 2013 and Pat. No. 8,632,786 issued Jan. 21, 2014 to the same inventor as the present inventor and which are incorporated herein by reference.

The above noted treatment can also be used for the treatment of

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What is claimed is:

1. A method for dosing botulinum toxin in a living human muscle of a patient for the treatment of plantar fasciitis comprising the steps of:
a) determining a target muscle in a selected calf muscle of the patient;
b) determining a target muscle and structure in a selected foot muscle of the patient;
c) injecting a dosing regimen comprising dose of botulinum toxin ranging from about 100 to about 180 units in a patient's selected calf muscle; and
d) injecting a dosing regimen comprising dose of botulinum toxin ranging from about 20 to about 90 units in selected foot muscles.

2. The method of claim 1 wherein said botulinum toxin is botulinum toxin A.

3. The method of claim 1 wherein said botulinum toxin is botulinum toxin B.

4. The method according to claim 1 wherein said botulinum toxin is selected from the group botulinum toxin serotypes consisting of botulinum toxin A, botulinum toxin B, botulinum toxin C, botulinum toxin D, botulinum toxin E and botulinum toxin F.

5. The method of claim 1 wherein said foot muscle dosage is unit base diluted in about 1.0 cc to about 3.0 cc of saline solution.

6. The method of claim 1 wherein said calf muscle dosage is diluted in a range of about 2.5 cc to about 8.0 cc of saline solution.

7. The method of claim 1 wherein said selected foot muscle is taken from the muscle group including plantar intrinsic muscles, plantar fascia and calcaneal periosteum.

8. The method of claim 1 wherein said selected calf muscle is taken from the muscle group gastrocnemius-soleus complex.

9. A method for dosing botulinum toxin in a living human muscle of a patient comprising the steps of:
a) determining a target muscle in the calf muscle of the patient;
b) determining a target muscle in the foot muscle of the patient;
c) injecting a dosing regimen comprising dose of botulinum toxin ranging from about 50 to about 200 units in a plurality of sites in a patient's calf, namely, the gastrocnemius-soleus complex; and
d) injecting a dosing regimen comprising dose of botulinum toxin ranging from about 20 to about 90 units in a patient's foot muscle group, namely, plantar intrinsic muscles, plantar fascia and calcaneal periosteum.

10. A method for dosing botulinum toxin as claimed in claim 12 wherein said foot muscle is the plantar intrinsic muscles.

11. A method for dosing botulinum toxin as claimed in claim 12 wherein said foot muscle is the plantar fascia.

12. A method for dosing botulinum toxin as claimed in claim 12 wherein said foot muscle is the calcaneal periosteum.

13. A method for dosing botulinum toxin in a living human muscle of a patient for the treatment of plantar fasciitis comprising the steps of:
a) selecting a target muscle in the calf muscle of the patient;
b) selecting a target muscle in the foot of said patient;
c) injecting a dosing regimen comprising a plurality of spaced doses of botulinum toxin ranging from about 100 units to about 160 units in a plurality of sites ranging in number from about 5 to about 7 depending on the leg length/mass and neuromuscular junctions distribution units in a patient's calf muscle; and
d) injecting a dosing regimen comprising a plurality of spaced doses of botulinum toxin ranging from about 40 units to about 80 units in said selected target foot muscle of said patient.

14. The method of claim 13 wherein said botulinum toxin is botulinum toxin A.

15. The method of claim 13 wherein said botulinum toxin is botulinum toxin B.

16. The method according to claim 1 wherein said botulinum toxin is selected from the group botulinum toxin serotypes consisting of botulinum toxin A, botulinum toxin B, botulinum toxin C, botulinum toxin D, botulinum toxin E and botulinum toxin F.

17. The method of claim 13 wherein said selected target foot muscle is taken from a group of foot muscles consisting of plantar intrinsic muscles, plantar fascia and calcaneal periosteum.

18. The method of claim 13 wherein said selected target calf muscle is taken from a group of calf muscles consisting of the gastrocnemius-soleus complex.

19. A method for dosing botulinum toxin in a living human muscle of a patient for the treatment of plantar fasciitis comprising the steps of:
a) determining a target muscle and structure in a selected foot muscle of the patient;
b) injecting a dosing regimen comprising dose of botulinum toxin ranging from about 100 to about 180 units in a patient's selected calf muscle; and
c) injecting a dosing regimen comprising dose of botulinum toxin ranging from about 20 to about 90 units in selected foot muscles.

20. The method of claim 19 wherein said botulinum toxin is botulinum toxin A.

21. The method of claim 19 wherein said botulinum toxin is botulinum toxin B.

22. The method according to claim 19 wherein said botulinum toxin is selected from the group botulinum toxin serotypes consisting of botulinum toxin A, botulinum toxin B, botulinum toxin C, botulinum toxin D, botulinum toxin E and botulinum toxin F.

23. A method as claimed in claim 19 wherein said treatment is the treatment of plantar fasciitis.

24. A method as claimed in claim 19 wherein said treatment is the treatment of intrinsic contracture.

25. A method as claimed in claim 19 wherein said treatment is the treatment of enthesopathy.
